(19) United States
(12) Patent Application Publication
(10) Pub. No.: US 2005/0154401 A1
(43) Pub. Date: Jul. 14, 2005

(54) Suturing device for implantable device

(75) Inventors: James Weldon, Roslindale, MA (US); Robert Rioux, Ashland, MA (US)

Correspondence Address: Joseph R. Kelly;Westman, Champlin & Kelly, Suite 1600, 900 Second Avenue South, Minneapolis, MN, 55402-3319, US

(73) Assignee: Scimed Life Systems, Inc., Maple Grove, MN (US)

(21) Appl. No.: 10/754,362

(22) Filed: Jan. 8, 2004

(51) Int. Cl. A61B 17/10

(52) U.S. Cl. 606/139

(57) ABSTRACT

A suturing device is used for fixing an implantable element in place. The suturing device includes an array of hypotubes, each of which includes a fastener for fastening the implantable device to the body, and an advancement device for advancing the fasteners through the array of tubes.

### BACKGROUND OF THE INVENTION

The present invention deals with suturing devices. More specifically, the present invention deals with a suturing device for securing a vascular graft or other implantable device.

There are a wide variety of uses for suturing devices for securing implantable medical devices in place. One environment in which a suturing device can be employed is in vascular graft placement. For example, the primary treatment options for ascending aortic aneurysms (AAA) include a conventional surgical graft procedure. The procedure is known as open surgical repair, or OSR. Another treatment option for AAA includes endovascular stent graft repair, also known as EVAR. The goal for both of these procedures is the same—to secure a vascular graft below the renal area in order to provide a new conduit for blood to the iliacs, such that the new conduit excludes the aneurysm from exposure to blood flow.

In the conventional OSR procedure, the proximal seal for the vascular graft is achieved with a surgical anastomosis. The anastomosis is simply formed by the surgeon suturing the graft in place. The EVAR procedure attempts to replicate an OSR anastomosis mechanically by using a stent frame in the vascular graft that includes external barbs which find purchase in the vessel in which the graft is placed. The long term integrity of the proximal seal for such stent grafts depends on the integrity of the stent and the barbs which are used.

Of course, this is but one environment in which suturing devices are used, and many other environments use suturing devices as well.

### SUMMARY OF THE INVENTION

A suturing device is used for fixing an implantable element in place. The suturing device includes an array of hypotubes, each of which includes a fastener for fastening the implantable device to the body, and an advancement device for advancing the fasteners through the array of tubes.

### BRIEF DESCRIPTION OF THE DRAWINGS

FIG. 1 illustrates an aneurysm in vasculature, such as in the ascending aorta.

FIG. 2 illustrates one embodiment of a device for treating the aneurysm shown in FIG. 1.

FIG. 3 is an illustration of a portion of a suturing device in accordance with one embodiment of the present invention.

FIG. 4A illustrates a more detailed portion of the suturing device shown in FIG. 3.

FIG. 4B shows another embodiment of a portion of the suturing device shown in FIG. 3.

FIG. 5 is an illustration of a portion of the suturing device inserted at a treatment site in accordance with one embodiment of the present invention.

FIG. 6A shows the suturing device with an exterior sheath and delivery catheter removed.

FIG. 6B is a cross-sectional diagram of the suturing device shown in FIG. 6A.

FIG. 7A illustrates the suturing device with tubes deployed in accordance with one embodiment of the present invention.

FIG. 7B shows the suturing device with attachment members deployed in accordance with an embodiment of the invention.

FIG. 7C illustrates removal of the suturing device once fixation components are in place.

FIG. 8 shows the suturing device with a centering balloon in accordance with one embodiment of the present invention.

FIG. 9A illustrates the suturing device with sutures and needles in accordance with another embodiment of the present invention.

FIG. 9B shows needles and suture in greater detail.

FIG. 9C shows removal of the suturing device once the needles and sutures are deployed.

FIG. 10 illustrates one embodiment of loading a graft for suturing.

### DETAILED DESCRIPTION OF THE PREFERRED EMBODIMENTS

While the present invention can be used in a wide variety of environments, it is described herein with respect to suturing a vascular graft for treating an ascending aortic aneurysm. For purposes of the description, proximal is used to mean a direction toward the treating physician while distal is used to mean a direction away from the treating physician.

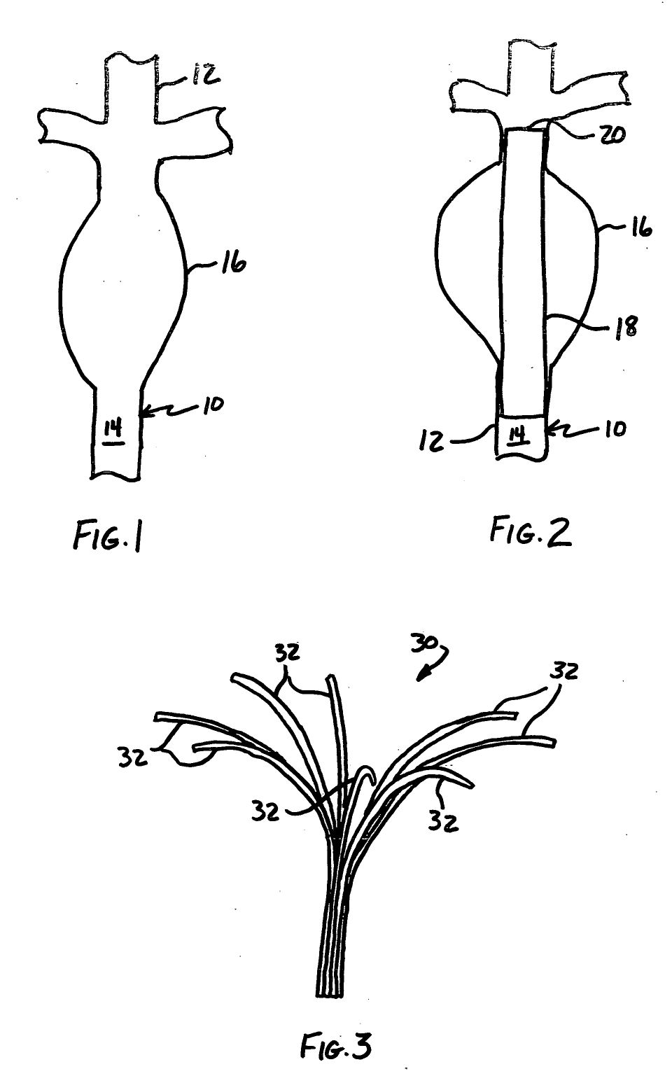

FIG. 1 illustrates a blood vessel 10 which includes a vessel wall 12 that defines a lumen 14. FIG. 1 also shows an aneurysm 16 in which vessel wall 12 is widened.

FIG. 2 shows one illustrative embodiment of an implantable device for treating aneurysm 16. In FIG. 2, a vascular graft 18 is disposed across aneurysm 16. In order to deploy graft 18, it is secured at a distal end 20 to vessel wall 12. This provides for blood flow through graft 18, across the site of vessel wall 12 that contains aneurysm 16, without exposing aneurysm 16 to the blood flow.

As described in the background portion hereof, the distal end 20 of graft 18 has illustratively been secured to vessel wall 12 to create an anastomosis in one of a number of different ways. One way simply includes a surgeon suturing the end 20 of graft 18 to vessel wall 12 to create the anastomosis. In another embodiment, a stent is included as part of end 20 of graft 18. The stent drives deployment of the end 20 of graft 18 to come into contact with the interior of vessel wall 12. The stent may illustratively include barbs that attempt to attach to vessel wall 12.

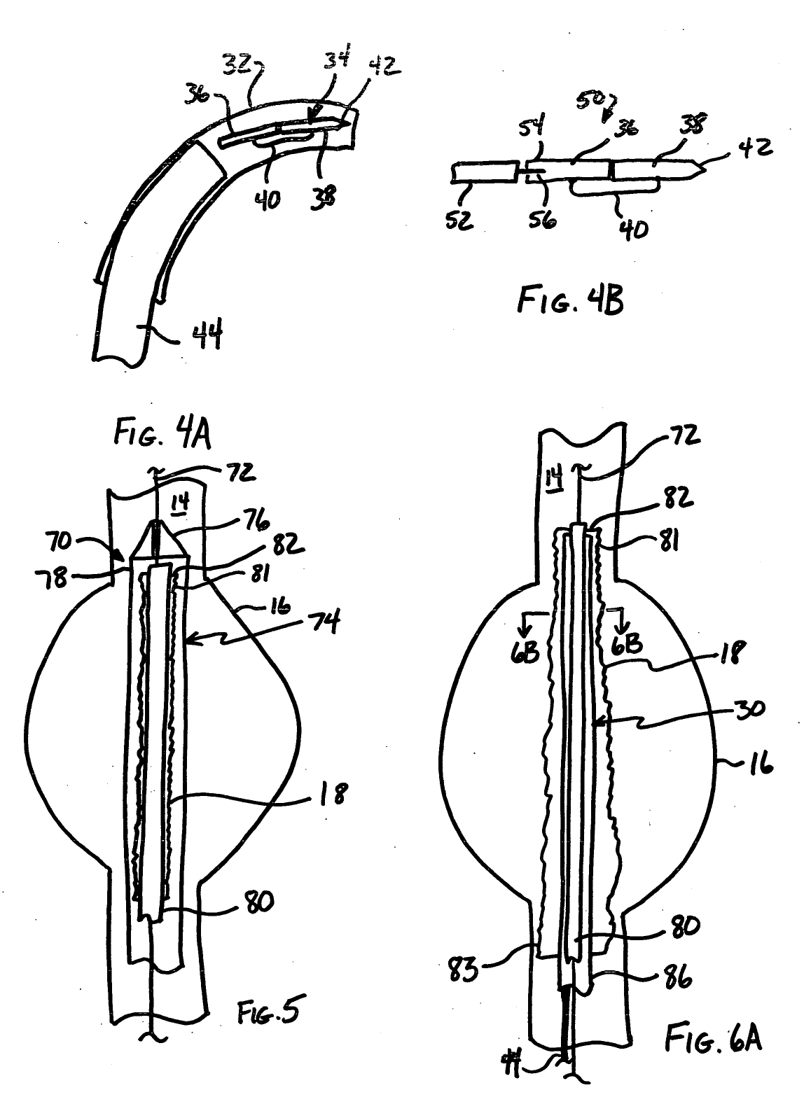

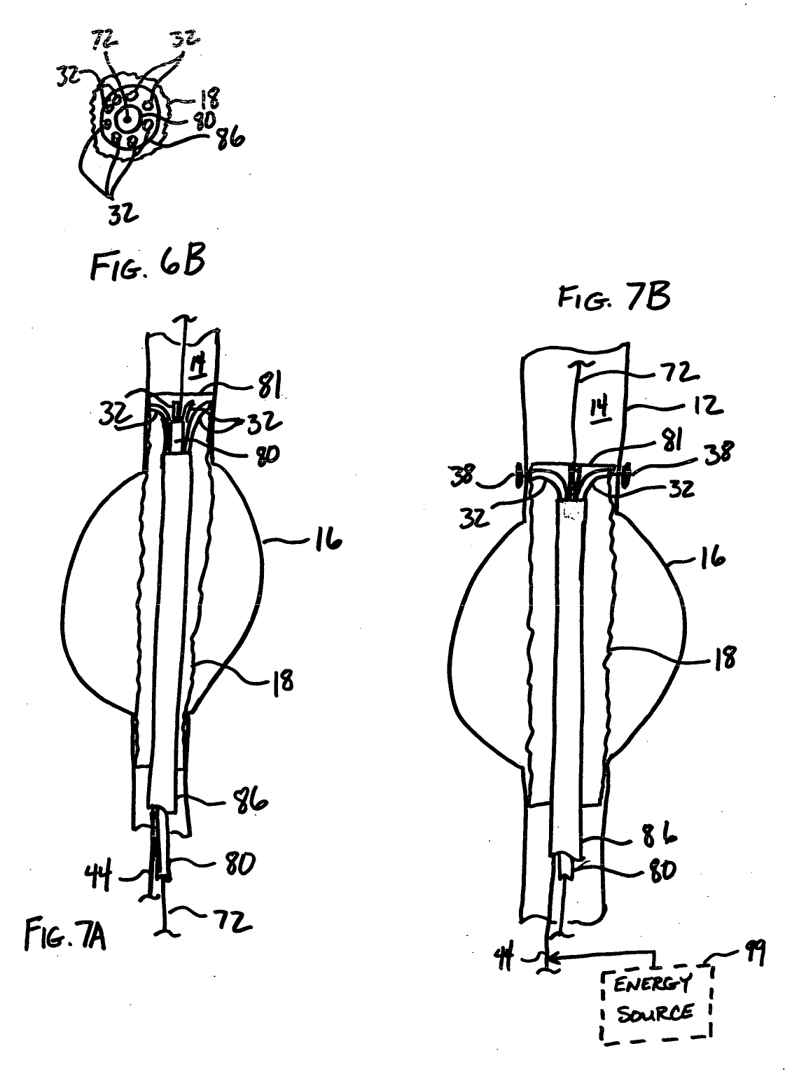

FIG. 3 illustrates one embodiment of a portion of a suturing device 30 in accordance with one embodiment of the present invention. Suturing device 30 includes a plurality of hollow hypotubes 32. Tubes 32 are illustratively resilient such that they can be biased into an elongate, generally longitudinal shape, such as by a delivery sheath. However, when tubes 32 are removed from the delivery sheath, the resilient hypotubes 32 extend radially outwardly to the position shown in FIG. 3. In one embodiment (as discussed in greater detail later with respect to FIGS. 5-7B), hypotubes 32 each have a plunger advancable therethrough to deploy fasteners or fixation elements through end 20 of vascular graft 18 fastening graft 18 to vessel wall 12.

FIGS. 4A and 4B illustrate two embodiments of a fixation element in more detail. FIG. 4A illustrates a distal end of one of hypotubes 32 in greater detail. In the distal end of hypotube 32 a T-fastener 34 is slidably disposed. T-fastener 34 includes a first fixation component 36, a second fixation component 38 and a tether 40 which is connected to components 36 and 38. In one illustrative embodiment, the distal end 42 of fixation component 38 is sharpened to pierce vascular graft 20 in vessel wall 12. FIG. 4A also illustrates a plunger or pusher member 44 disposed within tube 32 for advancing T-fastener 34 therethrough.

FIG. 4B illustrates another embodiment of a T-fastener 50 and pusher, or plunger 52. In the embodiment shown in FIG. 4B, T-fastener 50 is formed in a manner similar to that shown in FIG. 4A, and similar items are similarly numbered. However, an end 54 of fastener member 36 has a slot defined therein for receiving a tab 56 protruding from the pushing end of pusher member 52. In one embodiment, tab 56 simply frictionally engages the slot in member 54 to keep fastener 50 aligned with pusher 52 during deployment of T-fastener 50. In another embodiment, of course, tab 56 can be disconnectably connected to member 54, such as through a threadable engagement, an electrolytic disintegrating connection or any other type of disconnectable connection.

FIGS. 5-7B illustrate the operation of suture device 30 in accordance with one embodiment of the present invention. FIG. 5 is a more detailed diagram of a deployment system 70 for deploying suture device 30. FIG. 5 illustrates deployment system 70 in the vasculature at the site of aneurysm 16. FIG. 5 illustrates that deployment system 70 includes guidewire 72, and a delivery sheath 74 with a tip 76. FIG. 5 shows that outer sheath (or delivery sheath) 74 has been advanced over guidewire 72, which was itself advanced to the site of aneurysm 16. The end 78 of delivery sheath 74 is positioned just past aneurysm 16.

An inner sheath 80 defines an inner lumen which tracks over guidewire 72. The inner sheath 80 also has graft 18 wrapped thereabout. Graft 18 can be wrapped with a removable wrapper (not shown) to hold it in a radially contracted conformation about inner sheath 80. Graft 18 has an end 81 which is secured to the inner member 80 by a removable fixation suture 82. Suture 82 holds the end 81 of graft 18 tightly adjacent the outer surface of inner sheath 80 and extends proximally for removal by the physician. During insertion of the inner sheath 80, suture 82 is held tight to reduce a tendency for blood to be funneled into the interior of graft 18 through end 81.

Once in the position shown in FIG. 5, the wrapper disposed about graft 18 is removed and the delivery sheath 74 is also removed. This is illustrated in FIG. 6A. However, it should be noted that FIG. 6A shows that graft 18 still has the fixation suture 82 holding the end 81 of the graft 18 to the end of the inner sheath 80.

With the wrapper removed from graft 18 its proximal end 83 opens and suturing device 30 is tracked over inner sheath 80 and within graft 18 until its distal end reaches the retaining suture 82. FIG. 6A shows suturing device 30 in this position, and FIG. 6B is a cross-sectional diagram of suturing device 30 taken along section lines 6B-6B in FIG. 6A. FIG. 6B shows that suture device 30 has a delivery sheath 86 with the plurality of tubes 32 extending therethrough. FIG. 6B also shows that suture device 30 includes an inner lumen 88 sized to receive inner sheath 80 therein. Inner sheath 80 is also shown in FIG. 6B to have an internal lumen for receiving guidewire 72.

Again, it should be noted that tension is retained on fixation suture 82 until graft 18 is ready to be deployed by the suturing device 30. This reduces the windsock effect on graft 18. Therefore, as suture device 30 is tracked over inner sheath 80, hollow tubes 32 are retained in a generally longitudinal conformation by delivery sheath 86 such that they have a longitudinal axis that is generally parallel with the longitudinal axis of guidewire 72. It can be seen that, once suturing device 30 is in the position shown in FIG. 6A, it is generally coaxially located within graft 18.

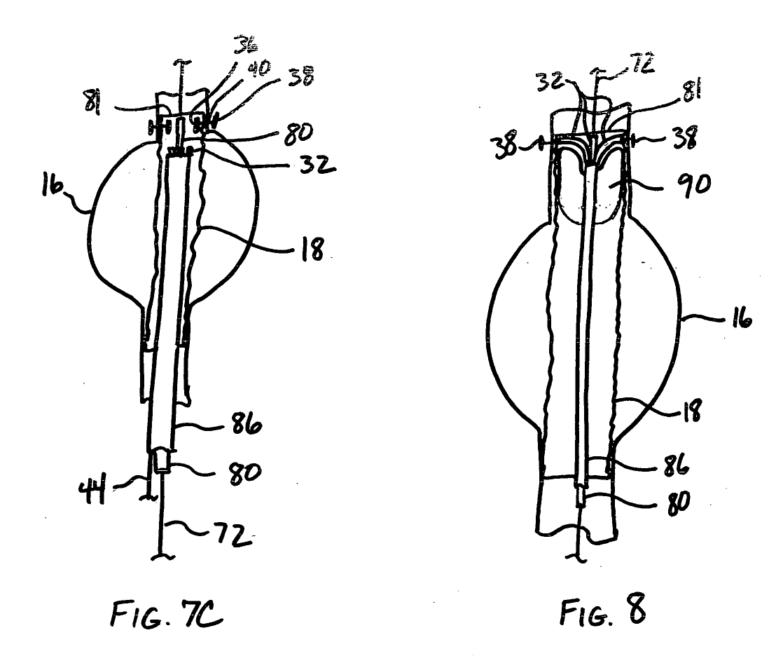

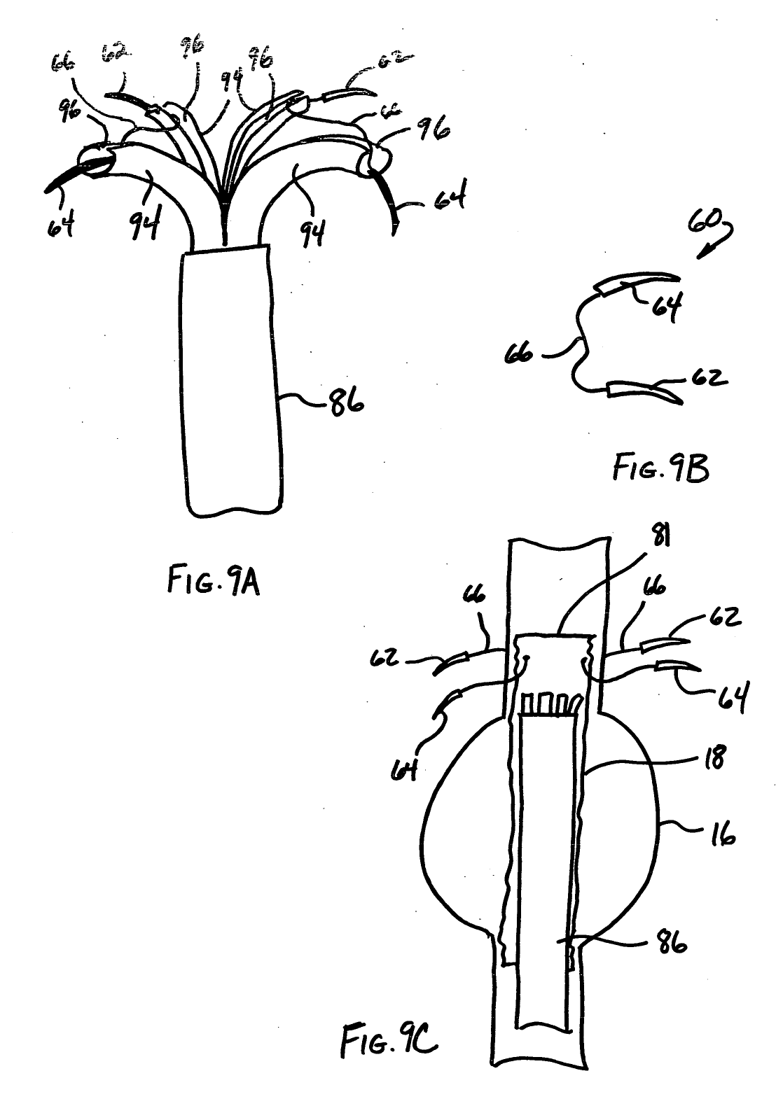

After suturing device 30 is advanced to the point shown in FIG. 6A, fixation suture 82 is removed and the tubes 32 of suturing device 30 are advanced from within delivery sheath 86. This can be done by advancing tubes 32 relative to delivery sheath 86 or by retracting delivery sheath 86 relative to the distal ends of tubes 32. In any case, the resilience of tubes 32 drives them to radially expand to drive the end 81 of graft 18 radially outwardly into frictional engagement with the inside of vessel wall 12 in lumen 14. Of course, prior to this, fixation suture 82 is removed, or it is removed simultaneously with the deployment of tubes 32 in the radially outward direction. Device 30 is illustrated in this position in FIG. 7A.

It will be noted that the distal end of each of the tubes 32 illustratively press the graft 18 evenly against the vessel wall. The distal ends of tubes 32 are illustratively blunted or squared off so the graft 18 and the vessel wall 12 are not punctured by the ends of the tubes 32.

Once tubes 32 are deployed as shown in FIG. 7A, the plungers 44 within each of tubes 32 are advanced to drive members 34 of T-fasteners 40 through the wall of graft 18, and through the vessel wall 12. This is shown in FIG. 7B. The tubes 32 are then retracted within delivery sheath 86. This causes the members 36 of T-fasteners 34 to exit the end of tubes 32 and to reside within the lumen formed by graft 18. Suture 40, of course, extends between members 36 and 38. Therefore, the T-fasteners are disposed in a position to secure the sheath 18 to the vessel wall 12 as shown in FIG. 7C. The entire system can then be removed from the vasculature, either in stages in which one or more elements are removed prior to other elements, or all at once.

Other embodiments of the present invention can be used as well. For example, FIG. 8 illustrates an inflatable member 90 that is disposed about the exterior portion of delivery sheath 86. In that embodiment, delivery sheath 86 illustratively includes an inflation lumen communicable with inflatable member 90. Inflatable member 90 can illustratively be inflated to drive radial expansion of end 81 of graft 18 to frictionally engage the inside of vessel wall 12. This ensures that graft 18 is expanded and held in the proper position during deployment of the tubes 32 of suturing device 30. This also ensures that sheath 86 is centered within the lumen and helps to ensure that tubes 32 are uniformly distributed about a periphery of sheath 86.

After inflation of inflatable device 90, tubes 32 are advanced from within delivery sheath 86. The plungers within tubes 32 are then advanced to deploy T-fasteners 34, and graft 18 is thus secured to the vessel wall 12.

In yet another embodiment, inflatable member 90 has an end 92 which is curved to have an outward conformation that generally corresponds to the conformation of tubes 32 in the radially expanded position. This allows inflatable member 90 to exert frictional pressure on graft 18 in the radial outward direction at a point closely proximate the tips of tubes 32, as they engage graft 18.

It should be noted that, while inflatable member 90 is illustratively made of compliant material (such as silicone or latex) it can be formed of non-compliant material as well. The particular material used may also vary with application (such as with the size of the vessel, etc.).

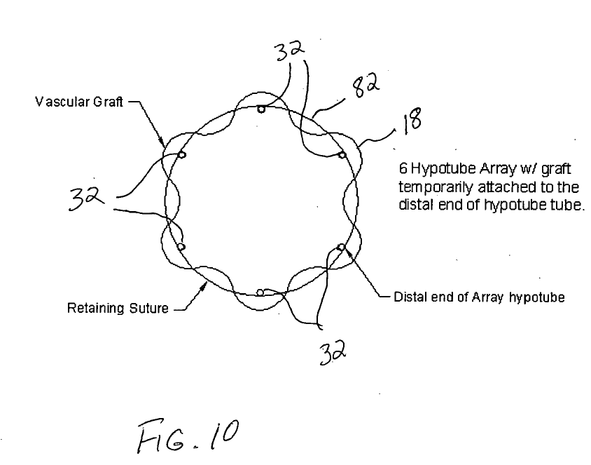

FIGS. 9A and 9B illustrate yet another embodiment. In the embodiment shown in FIGS. 9A and 9B, instead of tubes 32 being used, tubes 94 are provided. Tubes 94 are resilient in a fashion similar to tubes 32, and are deployed in a similar way. However, they also illustratively include longitudinal slots 96 therein. FIG. 9B shows that, instead of T-fasteners 34 or 50 (shown in FIGS. 4A and 4B), a pair of needles 62 and 64 are provided with suture 66 extending there between. Rather than two fastener members residing in each tube 94, only a single needle resides in each tube 94 and the suture 66 extends between the needles 62 and 64 in two adjacent tubes 94, and rides in slots 96 in tubes 94.

During deployment, graft 18 with its fixation suture is deployed in a similar fashion to that shown in FIG. 6A. Again, as shown in FIG. 7A, the suturing device is advanced over the inner sheath 80 and includes a delivery sheath 86. The tubes 94 are deployed from within sheath 86, instead of tubes 32. When the tubes 94 are removed from within delivery sheath 86, the plungers are advanced within tubes 94 and the needles 62 and 64 are advanced through graft 18 and through the vessel wall 12. This provides needles that have penetrated through the graft and the vessel wall, but have a piece of suture material 66 which remains within the vessel and graft and which is tethered to the two needles.

In a laproscopic procedure (or other procedure), a surgeon then ties a knot in the suture external to the vessel wall, using the suture attached to the needles and thus secures the graft 18 to the vessel wall. The two needles are illustrated in the deployed position, prior to the knot being tied, in FIG. 9C. While only two pairs of needles are shown in FIG. 9C, it will be appreciated that multiple additional pairs of needles may be provided, as desired by the physician in order to obtain a secured fixation of graft 18 to vessel wall 12.

Of course, the embodiment illustrated in FIGS. 9A-9C can also include the inflatable member 90 shown in FIG. 8 as well. Suture device 30 can include fewer tubes and they can simply be rotated about a longitudinal axis within graft 18 and re-deployed multiple times to secure graft 18 to the vessel wall.

In accordance with yet another embodiment of the present invention, the vessel wall 12 may be calcified. In that embodiment, pusher members (or plungers) 44 or 52 which travel through tubes 32 or 94 are formed of stainless steel or other radio frequency (RF) energy conducting material. A radio frequency energy source (99 in FIG. 7B) is provided external to the patient and RF energy is applied to the surface of the vessel though plungers 44 or 52 and the T-fastener (34 or 50) or needles 62 and 64. This allows the T-fastener or needles to more easily pierce calcified vessel wall.

It will also be noted that many different materials can be used for hypotubes and T-fasteners on needles described herein. NiTi and stainless steel are suitable examples, and biocompatible plastics biocompatible polymers, or other materials can be used as well.

It should also be clear that the present invention can be used in other applications as well. For instance, the device can be used for EVAR repair as well. Stent grafts can be reinforcably attached to the vessel wall to inhibit migration. Multiple T-fasteners can be deployed to help seal the proximal neck of the graft.

Similarly, to reduce the profile of the system, retaining suture 82 can be part of a separate graft delivery catheter. Such a catheter would illustratively be comprised of sheath 74, sheath 80 and fixation suture 82. In that embodiment, the delivery system is illustratively advanced to deploy the graft. The fixation suture 82 retains the graft in place, and the proximal end of the delivery catheter is dismantled leaving the inner sheath 80 and fixation suture 82 in place. The suturing device tracks over the inner sheath 80 to attach the graft to the vessel wall.

In yet another embodiment, the graft 18 can be loaded for delivery in such a way as to enhance the likelihood of uniform deployment of the graft. This is illustrated in FIG. 10. FIG. 10 shows that graft 18 is folded about tubes 32 and retained there by suture 82. The folds in graft 18 ensure that tubes 32 engage graft 18 at points equally spaced from one another about the graft periphery, when the array of tubes 32 is fully opened.

Although the present invention has been described with reference to preferred embodiments, workers skilled in the art will recognize that changes may be made in form and detail without departing from the spirit and scope of the invention.

1. A fixation system for fixing an implantable device in a body cavity, comprising:
a plurality of resilient delivery members movable between a generally longitudinal delivery position and a radially expanded deployment position, the delivery members defining a delivery channel therein with a distal opening;
a fixation component slidably disposed in each of the delivery channels; and
a pusher slidably disposed in each of the delivery channels to push the fixation component in each delivery channel.

2. The fixation system of claim 1 and further comprising:
a delivery sheath slidable over the plurality of resilient delivery members.

3. The fixation system of claim 1 wherein the delivery members define the delivery channel as a closed lumen therein with the distal opening.

4. The fixation system of claim 1 wherein each fixation component comprises:
a first fixation member;
a second fixation member; and
a tether connecting the first and second fixation members.

5. The fixation system of claim 4 wherein the delivery members, when in the deployed position, urge the implantable device against a wall of the body cavity.

6. The fixation system of claim 5 wherein the first fixation member is disposed to pierce the implantable device and a wall of the body cavity when advanced from the delivery channel by the pusher.

7. The fixation system of claim 6 wherein the first fixation member has a sharpened end for piercing the implantable device and body cavity wall.

8. The fixation system of claim 6 wherein the first and second fixation members are arranged in a generally longitudinally aligned orientation when in the delivery channel.

9. The fixation system of claim 8 wherein one of the first and second fixation members are releasably connected to the pusher.

10. The fixation system of claim 2 and further comprising:
an inner sheath, the plurality of delivery members being arranged generally radially about the inner sheath.

11. The fixation system of claim 10 wherein the implantable device comprises:
a vascular graft.

12. The fixation system of claim 11 and further comprising:
a releasable fixation member releasably fixing the vascular graft to a distal end of the inner sheath.

13. The fixation system of claim 10 and further comprising:
an expandable member expandable from a contracted position closely proximate an exterior of the delivery sheath to an expanded position urging the vascular graft against the wall of the body cavity.

14. The fixation system of claim 13 wherein the expandable member is positioned at a distal end of the delivery sheath.

15. The fixation system of claim 14 wherein the expandable member has a distal end thereof shaped in the expanded position to conform to a shape of the delivery members in the deployment position.

16. The fixation system of claim 2 wherein each of the delivery members define an associated delivery channel as a channel having a slot communicating with an exterior of the delivery member.

17. The fixation system of claim 16 wherein the fixation component comprises:
a piercing member with a tether attached thereto.

18. The fixation system of claim 17 wherein pairs of piercing members in adjacent delivery members are tethered together by the tether.

19. The fixation system of claim 18 wherein the tether is oriented to ride through the slots in the adjacent delivery members as the pushers advance the piercing members through the channel in the delivery members.

20. The fixation system of claim 19 wherein the pairs of piercing members are advanced through the implantable device and through a wall of the body cavity, the piercing members pulling ends of the tether through the implantable device and through the wall of the body cavity.

21. The fixation system of claim 1 and further comprising:
a radio frequency (RF) energy source connected to the pushers to apply RF energy to a wall of the body cavity through the pushers and the fixation components.

22. The fixation system of claim 1 wherein the body cavity comprises a vascular lumen in a region of an aneurysm.

23. The fixation system of claim 1 wherein the delivery members are configured to exert outwardly directed pressure on an inner wall of the implantable device at substantially uniformly spaced areas, when in the deployed position.

24. A method of fixing an implantable device to a wall of a body cavity, comprising:
advancing a plurality of resilient delivery members in the implantable device in the body cavity;
radially expanding the delivery members to urge the implantable device against the wall of the body cavity; and
advancing a fixation component from within each of the delivery members, a portion of each fixation component piercing the implantable device and the wall of the body cavity.

25. The method of claim 24 wherein radially expanding comprises:
removing the delivery members from within a delivery sheath, to allow the delivery members to move to a radially expanded position.

26. The method of claim 24 wherein advancing a fixation component comprises:
sliding a plunger in each of the delivery members to push the fixation components in the delivery members.

27. The method of claim 24 wherein advancing a plurality of resilient delivery members comprises:
advancing a tracking sheath to a treatment site in the body cavity; and
advancing the implantable device, along with the tracking sheath, to the treatment site.

28. The method of claim 27 wherein advancing a plurality of resilient delivery members comprises:
sliding the plurality of delivery members in tracking relation to the tracking sheath to the treatment site.

29. The method of claim 28 wherein the implantable device includes a vascular graft and wherein sliding the plurality of delivery members comprises:
unwrapping the vascular graft to allow radial expansion of an end of the vascular graft.

30. The method of claim 29 wherein sliding the plurality of delivery members comprises:
advancing the delivery members within the vascular graft.

31. The method of claim 30 wherein advancing the implantable device comprises:
maintaining a connection between the tracking sheath and a distal end of the vascular graft; and
releasing the connection prior to radially expanding the delivery members.

32. The method of claim 25 and further comprising:
prior to removing the delivery members from the delivery sheath, expanding an expandable member to urge the implantable device against the wall of the body cavity.

33. The method of claim 32 wherein the expandable member comprises an inflatable member and wherein expanding the expandable member comprises:
inflating the inflatable member to exert radially directed pressure on the implantable device against the wall of the body cavity.

34. The method of claim 32 wherein expanding an expandable member comprises:
expanding the expandable member into a shape conforming to a shape of the radially expanded delivery members.

35. The method of claim 24 and further comprising: applying radio frequency (RF) energy to the wall of the body cavity through the fixation component prior to piercing the implantable device.

36. The method of claim 24 wherein each delivery member includes an associated channel therethrough and a slot communicating from the channel to an exterior of the associated delivery member, and wherein advancing a fixation component comprises:
advancing pairs of needles, attached with a length of suture material, through the channels in adjacent delivery members.

37. The method of claim 36 wherein advancing pairs of needles comprises:
advancing the needles through the implantable device and the walls of the body cavity.

38. The method of claim 37 and further comprising:
tying ends of the suture material carried by the needles, external to the body cavity.

39. The method of claim 38 wherein tying comprises:
laparoscopically accessing a treatment site external to the body cavity.

40. The method of claim 38 wherein the delivery members comprise a plurality of pairs of delivery members and wherein advancing pairs of needles comprises:
advancing a plurality of pairs of needles through the channels.

41. The method of claim 40 wherein tying comprises:
tying a plurality of knots in a plurality of ends of suture material carried by the plurality of pairs of needles.

42. The method of claim 24 and further comprising:
loading the implantable device about the delivery members, prior to advancing.

43. The method of claim 42 wherein loading comprises:
folding the implantable device about the delivery members at substantially uniformly spaced locations along the inner periphery of the implantable device.

44. A fixation system for affixing a vascular graft to a wall of a blood vessel across an aneurysm, comprising:
an array of delivery tubes advancable through the graft and movable between a radially contracted position and a deployment position in which ends of the delivery tubes expand radially to urge the graft against the wall; and
a plurality of fixation components, one component slidably disposed in each delivery tube, the fixation components slidable out of the delivery tubes to connect the graft to the wall of the blood vessel.

45. The fixation system of claim 44 and further comprising:
a plurality of plungers, one plunger slidably disposed in each delivery tube to advance the fixation components out of the delivery tubes.

46. The fixation system of claim 44 and further comprising:
a delivery sheath slidable over the array of delivery tubes, the delivery sheath holding the delivery tubes in the contracted position and removal of the delivery sheath allowing movement of the delivery tubes into the deployment position.
